(19) United States
(12) Patent Application Publication
(10) Pub. No.: US 2023/0405264 A1
(43) Pub. Date: Dec. 21, 2023

(54) COMBINING VIBRO-TACTILE STIMULATION WITH A PSYCHOLOGICAL INTERVENTION

(71) Applicant: nemotec GmbH, Starnberg (DE)

(72) Inventors: Christian HAUPTMANN, Starnberg (DE); Nicole HAUPTMANN, Starnberg (DE)

(21) Appl. No.: 18/331,991

(22) Filed: Jun. 9, 2023

(30) Foreign Application Priority Data
Jun. 10, 2022 (DE) 10 2022 205 898.4

(51) Int. Cl. A61M 21/02

(52) U.S. Cl. CPC A61M 21/02; A61M 2021/0022

(57) ABSTRACT

An apparatus for providing vibro-tactile stimulation for use in combination with a psychological intervention includes a plurality of stimulation units, each adapted to provide vibro-tactile stimulation to a user upon activation, a receiver configured to receive instructions as part of the psychological intervention, and a controller configured to activate at least a subset of the plurality of stimulation units according to the instructions. A computer program is also provided for providing a psychological intervention for use in combination with an apparatus for providing vibro-tactile stimulation, comprising instructions which, when the program is executed by a computer, cause the computer to output instructions to the apparatus (as part of the psychological intervention. Furthermore, a system is also provided for providing vibro-tactile stimulation in combination with a psychological intervention, comprising such apparatus and such computer program or a separate device configured to execute such computer program.

### CROSS REFERENCE TO RELATED APPLICATION

Priority is claimed to German Patent Application No. DE 10 2022 205 898.4, filed on Jun. 10, 2022, which is hereby incorporated by reference herein.

### FIELD

The present invention relates to an apparatus for providing vibro-tactile stimulation for use in combination with a psychological intervention. It also relates to a computer program for providing a psychological intervention for use in combination with such apparatus. Furthermore, it relates to a system for providing vibro-tactile stimulation in combination with a psychological intervention, comprising such apparatus and such computer program or a separate device configured to execute such computer program, respectively.

### BACKGROUND

It is generally known that vibro-tactile stimulation can be used to reduce pathological neuronal activity, which may result, inter alia, from Parkinson's disease, depression, anxiety disorders and/or burn-out. Likewise, it can be expected that vibro-tactile stimulation can be used to enhance healthy and beneficial neuronal activity, e.g., cognition.

Irrespective, at least some of the mentioned illnesses and/or diseases are usually treated by psychological intervention. One particular form of psychological intervention is Mindfulness Based Cognitive Therapy (MBCT). MBCT was originally developed—by Zindel Segal, Mark Williams and John Teasdale—to treat recurrent depression. But it can also be used for treatment of numerous other illnesses and/or diseases, e.g., to reduce stress or to uncover and change pathogenic thought patterns. At the same time, it involves learning valuable coping strategies for everyday life. In the process, the old value system is questioned and more helpful ways of thinking are offered. Another particular form of psychological intervention is stress reduction through relaxation training (e.g., relaxation exercises), e.g., as part of a rehabilitation program.

Even though the above treatments have each already proven effective, there is of course a continuous need to improve thereon, in turn rendering treatment of at least some of the mentioned illnesses and/or diseases still more effective.

### SUMMARY

In an embodiment, the present disclosure provides an apparatus for providing vibro-tactile stimulation for use in combination with a psychological intervention. The apparatus includes a plurality of stimulation units, each adapted to provide vibro-tactile stimulation to a user upon activation, a receiver configured to receive instructions as part of the psychological intervention, and a controller configured to activate at least a subset of the plurality of stimulation units according to the instructions. A computer program is also provided for providing a psychological intervention for use in combination with an apparatus for providing vibro-tactile stimulation, comprising instructions which, when the program is executed by a computer, cause the computer to output instructions to the apparatus (as part of the psychological intervention. Furthermore, a system is also provided for providing vibro-tactile stimulation in combination with a psychological intervention, comprising such apparatus and such computer program or a separate device configured to execute such computer program.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is an illustration of an exemplary embodiment of an apparatus for providing vibro-tactile stimulation for use in combination with a psychological intervention according to the present invention;

FIG. 2 is an illustration of a pattern according to which one or more of a plurality of stimulation units of an apparatus for providing vibro-tactile stimulation for use in combination with a psychological intervention according to the present invention may be activated; and

FIGS. 3A to 3H are an illustration of another exemplary embodiment of an apparatus for providing vibro-tactile stimulation for use in combination with a psychological intervention according to the present invention.

### DETAILED DESCRIPTION

Embodiments of the present invention provide to improve the treatments at least in part by an apparatus for providing vibro-tactile stimulation for use in combination with a psychological intervention. Such apparatus comprises a plurality of stimulation units, each adapted to provide vibro-tactile stimulation to a user upon activation, means for receiving instructions as part of the psychological intervention, in particular, a receiver, and means for activating at least a subset of the plurality of stimulation units according to the instructions, in particular, a controller. Put differently, the apparatus combines vibro-tactile stimulation with psychological intervention. More specifically, it coordinates and/or synchronizes vibro-tactile stimulation and a (simultaneous) psychological intervention. To this very end, the apparatus comprises said means for receiving instructions as part of the psychological intervention as well as said means for activating at least a subset of the plurality of stimulation units according to the instructions: These means allow the apparatus to activate at least said subset of the plurality of stimulation units according to instructions received as part of the psychological intervention. Thereby, the apparatus may coordinate and/or synchronize the activation of one or more stimulation units with one or more instructions forming part of the psychological intervention.

The inventors have found that such combination of vibro-tactile stimulation and psychological intervention yields synergistic effects. That is, it greatly enhances the therapeutic effect as compared to using vibro-tactile stimulation and/or psychological intervention separately and/or independently of each other, especially where the psychological intervention comprises elements of MBCT and/or relaxation training (e.g., relaxation exercises). The combination of vibro-tactile stimulation and psychological intervention may facilitate and intensify learning effects, as, e.g., MBCT exercises and/or relaxation trainings (e.g., relaxation exercises) are reinforced by parallel application of vibro-tactile stimulation. Furthermore, it may enhance the overall experience caused and/or invoked by the psychological intervention. This can be realized, e.g., by providing suitable vibro-tactile stimulation corresponding to the content of the psychological intervention. For example, vibro-tactile stimulation may be used to invoke the sensation of a breeze where the psychological intervention invokes the sound of the sea or wind. This can greatly increase the sensation and thus also the desired effect of relaxation. Likewise, e.g., during a breathing exercise, suitable, corresponding vibro-tactile stimulation of the abdominal region of the user can increase the desired effect. More generally, mindfulness and/or concentration and/or relaxation exercises may be significantly intensified in their effect by parallel application of vibro-tactile stimulation. The haptic experience makes it easier to stay focused on the exercise instead of letting one's thoughts quickly drift away.

The combination of vibro-tactile stimulation and psychological intervention may have numerous additional desirable effects. For example, after having used the apparatus and the combination of vibro-tactile stimulation and psychological intervention, respectively, a few times, conditioning may set in. For example, if the psychological intervention comprises elements of MBCT and/or relaxation training (e.g., relaxation exercises), in a later application, vibro-tactile stimulation alone may trigger sensations that were originally due to the respective elements/exercises.

The psychological intervention may be provided by a computer program. Additionally, the means for receiving the instructions may then be adapted to receive the instructions from the computer program. Providing the psychological intervention by a computer program renders the user independent of any third parties, such as a psychologist or psychiatrist. Rather, if the psychological intervention is provided by a computer program, the user may initiate the psychological intervention—and the corresponding vibro-tactile stimulation—all by him-/herself, at any time and/or at any location, e.g., at home. This greatly increases user-friendliness and may moreover render use of the apparatus less costly in that no fees may be due to a third party, such as a psychologist or psychiatrist. If the means for receiving the instructions are adapted to receive the instructions from the computer program, it may in particular receive the instructions in digital form. While it is generally possible that the means for receiving is adapted to receive the instructions in audio form (e.g., in the shape of instructions spoken by a third party, such as a psychologist or psychiatrist, or in the shape of instructions output by the computer program using a speaker) and to then transform them to digital form, e.g., by using voice recognition, it may be advantageous to receive the instructions from the computer program in digital form straight away as this may minimize errors and/or malfunctions.

In a preferred embodiment, the computer program is executed by a separate device and the means for receiving the instructions is adapted to receive the instructions from the separate device. Executing the computer program by a separate device may be useful to give a third party, such as a psychologist or psychiatrist, at least some control over the vibro-tactile stimulation and/or the psychological intervention. Also, this may allow to keep the apparatus as simple as possible; in particular, the apparatus itself does then not need any means to execute the computer program. This may in turn render the apparatus less costly, less prone to errors and/or malfunctions, and/or easier to repair.

The separate device may be a computer, e.g., a computer the user has at home. Preferably, the separate device may be a smartphone. The computer program may then also be available as, or be termed, an application, or app. Using a smartphone as the separate device has numerous advantages. Smartphones are ubiquitous and widely available, obviating the need for a dedicated separate device. This renders use of the apparatus even less costly and, at the same time, even more user-friendly given that the user is used to operating his/her smartphone as opposed to operating a dedicated separate device. Moreover, smartphones are usually equipped with various interfaces that may be used to communicate with the apparatus. For example, if the separate device is a smartphone, the means for receiving the instructions may preferably be adapted to receive the instructions from the smartphone using wireless communications, in particular Bluetooth and/or Wi-Fi.

Yet, the apparatus may also comprise means for executing the computer program itself, in particular, a processor, as well as means for providing the psychological intervention to the user. Then, the apparatus is self-contained, providing an all-in-one solution for combining vibro-tactile stimulation with a psychological intervention. Some may find this more user-friendly than operating both the apparatus as well as a separate device as just described, even where the separate device is a smartphone. In the same vein, such configuration of the apparatus may obviate any connection issues that could otherwise occur when trying to establish a connection, in particular a wireless connection, between the apparatus and any separate device. Furthermore, such configuration of the apparatus may facilitate troubleshooting in that it minimizes the number of sources for any errors and/or malfunctions.

Irrespective, the apparatus according to an embodiment of the present invention may also generally be capable of receiving instructions from a computer program that does not provide a psychological intervention. Such computer program may, e.g., be used to guide the user in using the apparatus for providing vibro-tactile stimulation without receiving a psychological intervention at the same time. For example, the user may use such computer program to turn on the apparatus or its stimulation units, respectively. Additionally or alternatively, such computer program may be used as a timer, aiding in applying vibro-tactile stimulation. Additionally or alternatively, such computer program may offer to play music while the user is undergoing vibro-tactile stimulation using the apparatus. Thereby, such computer program can support the user in applying the recommended amount of stimulation, e.g., per day, which might be one hour, or several hours. Furthermore, such computer program can keep track of total usage time(s) in order to provide feedback to the user which might increase his/her compliance. It is readily understood that all these functions may likewise additionally be performed by a computer program for providing a psychological intervention as described herein.

The apparatus may further comprise means for establishing which stimulation units of the plurality of stimulation units are in contact with the user, in particular, a sensor, wherein the means for activating is adapted to activate at least a subset of the plurality of stimulation units that consists of stimulation units that are established to be in contact with the user. This may ensure that only such stimulation units are activated that are in fact in contact with the user, i.e., that only such stimulation units are activated that may actually apply vibro-tactile stimulation to the user. This may allow to optimize vibro-tactile stimulation and the pattern(s) in which it is applied, respectively, in turn improving effectiveness of the treatment as a whole. Also, this may render the apparatus more efficient, in particular more energy efficient, in that no stimulations units are activated that are not in contact with the user and that may hence have no, or at least less, effect on him/her as regards vibro-tactile stimulation.

The apparatus may further comprise means for associating different stimulation units of the plurality of stimulation units with different parts and/or regions, in particular body parts and/or body regions, of the user. To this end, the means for associating may preferably comprises means for establishing a position and/or an orientation of the apparatus. Associating different stimulation units of the plurality of stimulation units with different parts and/or regions, in particular body parts and/or body regions, of the user may allow to further enhance the experience and hence the effectiveness of the treatment. More specifically, this may allow to even better combine, i.e., coordinate and/or synchronize, the vibro-tactile stimulation with the psychological intervention. In particular, different instructions provided as part of the psychological intervention may be associated with different stimulation units of the plurality of stimulation units and hence with different parts and/or regions, in particular body parts and/or body regions, of the user. Sticking with the example already mentioned above, if the instruction relates, e.g., to a breathing exercise, this instruction may be associated with the abdominal region of the user and suitable, corresponding vibro-tactile stimulation thereof may then be provided by activating the associated stimulation units. Likewise, a psychological intervention invoking a stimulus wandering across different parts and/or regions, in particular body parts and/or body regions, of the user may be associated with the corresponding stimulation units of the plurality of stimulation units that allow to physically trace the stimulus across different parts and/or regions, in particular body parts and/or body regions, of the user.

The apparatus may comprise a surface layer covering at least a portion of the apparatus. This surface layer may be removable. This may facilitate maintenance and/or cleaning of the apparatus as well as of the surface layer itself. To the same end, the surface layer may, additionally or alternatively, comprises a rinseable material. Further additionally or alternatively, the surface layer may comprise a skin-friendly material in order to enhance the user experience and to avoid any irritation of the user and his (body) parts and/or regions, respectively. A particularly suitable material fulfilling at least some of these requirements is silicone.

The apparatus may comprise an energy storage (rather than, e.g., a mains plug). This may render the apparatus mobile, making it even easier to use it at any time and/or at any location, e.g., while travelling. This does not only generally increase user-friendliness. This may also be advantageous considering that using the device may be desirable, or even necessary, when and where there is no external energy source, e.g., no mains socket, available, such as when a user suffering from acute flight anxiety is aboard an airplane. Preferably, the energy storage allows the apparatus to be used for approximately four to six hours. The energy storage may be replaceable, facilitating maintenance and allowing to minimize downtime by using two or more energy storages in alternation. Additionally or alternatively, the energy storage is rechargeable for the sake of sustainability. Especially if the energy storage is not replaceable, it may be rechargeable using inductive charging as this obviates the need for a corresponding connection (e.g., a socket) at the apparatus, in turn rendering it more user-friendly and, possibly, more sturdy. In particular, using inductive charging rather than a wired connection (that in turn requires a connection, e.g., a socket) at the apparatus, may facilitate making the apparatus waterproof. This may not only enhance the apparatus' longevity. It may also allow to use the apparatus, e.g., in wet environments, e.g., in the bathtub, which may add to the desired effect and experience to be invoked by the apparatus.

The apparatus may comprise between 2 and 500 stimulation units, preferably between and 400 stimulation units, more preferably between 10 and 300 stimulation units, most preferably 250 stimulation units. Additionally or alternatively, the stimulation units of the plurality of stimulation units may be arranged at an average distance of 0.1 cm to 20 cm, preferably at an average distance of 0.25 cm to 10 cm, more preferably at an average distance of cm to 2.5 cm, most preferably at an average distance of less than 1 cm. The inventors have found that such configurations of the stimulation units are the most versatile and/or useful.

In a preferred embodiment, the apparatus has a generally spherical shape with a diameter between 5 cm and 100 cm, preferably between 10 cm and 50 cm, more preferably between 20 cm and 25 cm. The inventors have found that such configurations of the apparatus are most versatile and/or useful. Especially when the apparatus has a generally spherical shape with a diameter between 20 cm and 25 cm, it is highly mobile (i.e., it can be taken virtually anywhere) and may be held by the user in one hand or in both hands (such that it may also be held against virtually any other part and/or region, in particular body part and/or body region, of the user; it may, e.g., also be placed beneath one foot or both feet of the user if desired), while still being large enough to house a sufficient number of stimulation units that allow for more elaborate and/or effective vibro-tactile stimulation.

If the apparatus has a generally spherical shape, it may optionally comprise at least one portion allowing the apparatus to be placed on a flat surface. This may be particularly advantageous if the apparatus has a larger diameter such that it may not easily be held by the user in one hand or in both hands; the flat surface may then act as a support for the apparatus, which may then still have a generally spherical shape, which has been found most versatile and/or useful by the inventors. Irrespective of the apparatus' size, such a portion allows to securely stow away the apparatus, especially when not in use.

Besides, such a portion may additionally be used to provide a (physical) user interface. For example, the portion and/or the user interface, respectively, may comprise an on/off switch. It may also comprise a connection (e.g., a socket or plug) for connecting the apparatus to an energy supply (e.g., a mains socket). Likewise, it may comprise a connection (e.g., a socket) for charging the apparatus or its energy storage, respectively; where the apparatus and/or the energy storage allow for inductive charging, said portion may be indicative of how to place the apparatus relative to an inductive charging device (e.g., a coil of the apparatus to be used for inductive charging may be arranged within and/or beneath said portion). Furthermore, the portion and/or the user interface, respectively, may comprise, where applicable, means for connecting, or means for configuring a connection of, the apparatus to a separate device. Such means for connecting may comprise a socket and/or a plug, especially in case of a wired connection to the separate device. Such means for configuring a connection may comprise one or more buttons, especially in case of a wireless connection to the separate device, which may, e.g., be a smartphone as described above. In an embodiment, such means for configuring a connection may comprise a button that initiates or allows establishing a wireless connection, e.g., via Bluetooth and/or Wi-Fi (commonly termed a pairing button). Furthermore, the portion and/or the user interface, respectively, may comprise one or more indicators, e.g., one or more light-emitting diodes (LEDs) to provide information about the apparatus' status. Yet, in other embodiments (in which the apparatus may however still have a generally spherical shape and optionally comprise at least one portion allowing the apparatus to be placed on a flat surface), such a user interface or any parts thereof may be arranged elsewhere.

The apparatus may be provided in the shape of a pillow. That is, the apparatus may be flexible and/or soft and suitable to be placed beneath a part and/or region, in particular a body part and/or body region, of the user. This may provide for a particularly comfortable and/or relaxing user experience, e.g., while lying down, e.g., in bed, which may in turn further add to the desired effect and experience to be invoked by the apparatus.

The apparatus may also comprise one or more recesses adapted to receive at least a tip of a finger and/or thumb of the user, wherein each of the one or more recesses comprises at least one stimulation unit of the plurality of stimulation units. Such one or more recesses may generally provide the apparatus with an ergonomic shape, allowing for quick and comfortable alignment of the user and his/her (body) part(s) and/or region(s) with the apparatus and the stimulation units. They ensure that the hands, or at least the tip of at least one finger and/or thumb, of the user is/are optimally placed on the apparatus, in turn also guaranteeing (optimal) contact of the respective stimulation unit arranged in the one or more recesses with (the tip of the respective finger and/or thumb of) the user. Such one or more recesses may moreover also predetermine which one or more stimulation units of the plurality of stimulation units will be in contact with which (body) part and/or region of the user. This makes associating different stimulation units of the plurality of stimulation units with different parts and/or regions, in particular body parts and/or body regions, of the user particularly easy.

The apparatus may also be at least partially hollow, and optionally at least partially flexible, such that it may be placed over another object. This renders the apparatus particularly versatile, not least increasing its range of application. If configured as described, the apparatus may be placed over various different objects, which may have any suitable shape and/or haptic characteristics depending on the desired effect and experience to be invoked by the apparatus. For example, if vibro-tactile stimulation is to be applied to a hand of the user, the apparatus may be placed over a suitably sized ball or a grip-shaped object. If vibro-tactile stimulation is to be applied to the back and/or neck of the user, the apparatus may be placed over a suitably sized pillow.

The apparatus may be provided in the shape of a glove. One or more, or even all, of the plurality of stimulation units may be arranged on the inside of the glove (i.e., the side and/or surface facing and/or touching the user's hand), on the outside of the glove (i.e., the side and/or surface facing away from the user's hand), or both. The plurality of stimulation units may be evenly or unevenly distributed over the surface of the glove. For example, the fingertips and/or the palm of the glove may comprise more stimulation units than the rest of the glove. Providing the apparatus in the shape of a glove makes it particularly easy and comfortable to use. For example, if one or more, or even all, of the plurality of stimulation units are arranged on the inside of the glove, the user does not need to (consciously) grab the apparatus for providing vibro-tactile stimulation. On the other hand, for example, if one or more, or even all, of the plurality of stimulation units are arranged on the outside of the glove, the user may easily guide the apparatus to different (body) parts and/or regions of the user.

The apparatus and/or its means for activating, respectively, may be adapted to activate one or more of the plurality of stimulation units according to one or more different patterns, which may be used and/or at least in part repeated, at least in part in isolation, combination and/or alternation, at least in part sequentially and/or simultaneously. Where more than one pattern is used, the patterns and/or their sequence, if any, may be chosen at least in part systematically and/or at least in part randomly. The inventors have found that the following patterns may provide for particularly desirable effects and experiences:

The means for activating may be adapted to activate at least the subset of the plurality of stimulation units synchronously, preferably at a frequency in the range of 1 Hz to 50 Hz, more preferably at a frequency in the range of 2 Hz to 20 Hz, most preferably at a frequency of 5 Hz, such as to generate a pulsing sensation for the user.

The means for activating may also be adapted to activate at least one or more subsets of the plurality of stimulation units throughout a plurality of bursts. Within such bursts, stimulation units may preferably be activated at a frequency in the range of 10 Hz to 300 Hz, more preferably at a frequency in the range of 20 Hz to 200 Hz, most preferably at a frequency in the range of 50 Hz to 90 Hz. Between such bursts, there preferably lapse 100 ms to 2000 ms, more preferably 150 ms to 1000 ms, most preferably 200 ms to 300 ms.

The means for activating may also be adapted to successively activate the stimulation units of at least the subset of the plurality of stimulation units, individually and/or in groups of two or more stimulation units, such as to generate the sensation of a wave travelling across the apparatus for the user.

The means for activating may also be adapted to activate the stimulation units of at least the subset of the plurality of stimulation units, individually and/or in groups of two or more stimulation units, at N respective onset times T₁ . . . T_{N} throughout a cycle period T, wherein N is an integer larger than one and wherein the onset times T₁ . . . T_{N} are not arranged substantially uniformly throughout the cycle period T. That is, the onset times T₁ . . . T_{N} are especially not arranged in uniform intervals substantially equal to T/N throughout the cycle period T.

Therein, the cycle period T may define the duration of the respective pattern, e.g., after a duration equal to cycle period T has lapsed, the same or a different pattern begins again. Notably, the subsequent pattern may be completely identical, i.e., it may define that the means for activating is adapted to activate the exact same stimulation units, individually and/or in the exact same groups of two or more stimulation units as before, at the exact same N respective onset times T₁ . . . T_{N} throughout the cycle period T. The subsequent pattern may however also differ in at least one respect. For example, it may define that the means for activating is adapted to activate the exact same stimulation units, individually and/or in the exact same groups of two or more stimulation units as before, but at different onset times T₁ . . . T_{N} throughout the cycle period T. This includes the case that the exact same onset times T₁ . . . T_{N} throughout the cycle period T are used, wherein however different stimulation units are associated with a given onset time than before. Likewise, the subsequent pattern may additionally or alternatively differ in the (number and/or grouping of) stimulation units the means for activating is adapted to activate, etc. The subsequent pattern may also differ completely, i.e., it may be one of those described above.

Additionally or alternatively, the cycle period T may be defined as a (longest) time period within which each of the stimulation units of at least the subset of the plurality of stimulation units is activated exactly once. The cycle period may also be defined as a duration between two consecutive onset times at which a same stimulation unit of at least the subset of the plurality of stimulation units is activated.

The above-described need is furthermore addressed at least in part by a computer program for providing a psychological intervention. The computer program is suitable for use in combination with an apparatus for providing vibro-tactile stimulation, such as that described hereinabove, and accordingly comprises instructions which, when the program is executed by a computer, cause the computer to output instructions to the apparatus as part of the psychological intervention. Reference is made to the above-described advantages regarding the use of the apparatus as described herein with a computer-program providing psychological intervention, outputting corresponding instructions to the apparatus as part of the psychological intervention.

Likewise, the above-described need is addressed at least in part by a system for providing vibro-tactile stimulation in combination with a psychological intervention which comprises an apparatus and a computer program, as described herein, or a separate device configured to execute such a computer program.

Finally, the above-described need may also be addressed at least in part by a method for providing vibro-tactile stimulation in combination with a psychological intervention. Such method may comprise giving instructions to a user as part of the psychological intervention and activating at least a subset of a plurality of stimulation units, each adapted to provide vibro-tactile stimulation to the user upon activation, according the instructions.

For the sake of brevity only a few embodiments will be described in the following. The skilled person will recognize that the specific features described with reference to these embodiments may be modified and combined differently and that individual features may also be omitted if they are not essential. The general explanations in the sections above will also be valid for the following more detailed explanations.

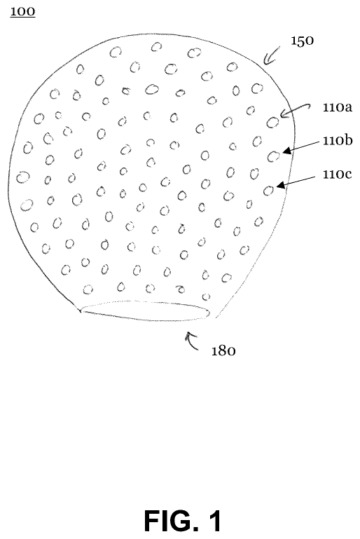

FIG. 1 shows an embodiment of the apparatus according to the present invention. The shown apparatus 100 has a generally spherical shape with a diameter of about 20 cm. It comprises about 250 stimulation units 100a, 110b, 110c (not all shown, let alone labelled) arranged uniformly at an average distance of about 1 cm. Apparatus 100 comprises a means for receiving instructions as part of a psychological intervention from a smartphone using Bluetooth. The smartphone correspondingly executes a computer program—i.e., an application, or app—for providing a psychological intervention according to an embodiment of the present invention that accordingly causes the smartphone to output instructions to apparatus 100 as part of the psychological intervention. Apparatus 100 comprises means for activating at least a subset of the plurality of stimulation units 110a, 110b, 110c according to these instructions. Preferably, the psychological intervention and/or the instructions provided as part thereof, respectively, comprises elements of MBCT and/or relaxation training (e.g., relaxation exercises).

Apparatus 100 comprises a portion 180 that allows apparatus 100 to be placed on a flat surface. This portion also comprises (e.g., houses and/or provides) a (physical) user interface. This user interface comprises an on/off switch, a Bluetooth pairing button, and an LED indicative of the apparatus' status. Portion 180 is also indicative of how to place apparatus 100 relative to an inductive charging device because a coil of apparatus 100 to be used for inductive charging is arranged beneath portion 180. That is, if apparatus 180 is placed on an inductive charging device such that portion 180 rests on said inductive charging device, apparatus 100 or its rechargeable energy storage, respectively, will be charged inductively.

Apparatus 100 further comprises a surface layer 150 covering apparatus 100 in full, except for portion 180. Surface layer 150 is made from silicone, i.e., it is rinseable and skin-friendly. Moreover, surface layer 150 is thus flexible and in turn removable.

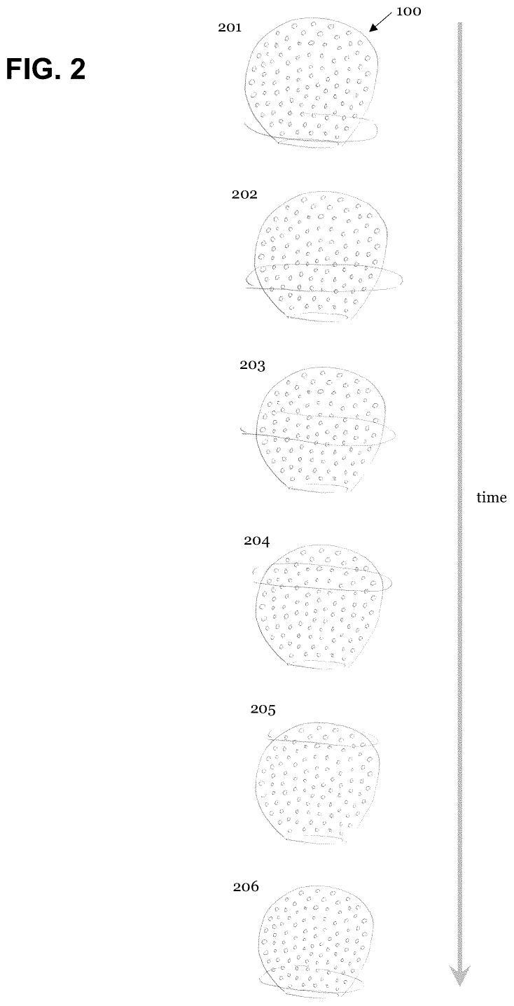

FIG. 2 shows a particular pattern according to which (a subset of) the stimulation units 110a, 110b, 110c of apparatus 100 may be activated. As shown, the means for activating of apparatus 100 is adapted to successively activate the stimulation units 110a, 110b, 110c in layered groups, such as to generate the sensation of a wave travelling across the apparatus 100 in upward direction (from bottom to top) for the user: First (panel 201), only a lowermost layer, i.e., a group of stimulation units 110a, 110b, 110c that comprises those stimulation units 110a, 110b, 110c that are closest to portion 180 of apparatus 100, is activated. Then, the layer immediately atop the lowermost layer, i.e., a group of stimulation units 110a, 110b, 110c that comprises those stimulation units 110a, 110b, 110c that are second closest to portion 180 of apparatus 100, is activated, and so forth, until (panel 205) the topmost layer, i.e., a group of stimulation units 110a, 110b, 110c that comprises those stimulation units 110a, 110b, 110c that are closest to the top of apparatus 100, is activated. Some intermediate layers are illustrated in panels 202 through 204. Then, the pattern begins anew (panel 206), such as to generate the sensation of another wave travelling across the apparatus 100 in upward direction for the user.

Several modifications to this pattern—as well as completely different patterns, such as those described above—are possible. For example, the means for activating of apparatus 100 may be adapted to successively activate the stimulation units 110a, 110b, 110c in layered groups, such as to generate the sensation of a wave travelling across the apparatus 100 from back (e.g., closest to the user when holding apparatus 100) to front (e.g., furthest away from the user when holding apparatus 100) rather than from bottom to top. The means for activating of apparatus 100 may also be adapted to successively activate the stimulation units 110a, 110b, 110c in layered groups, such as to generate the sensation of two waves travelling across one hemisphere of the apparatus 100, respectively, the waves preferably travelling in opposite directions. This may provide particularly desirable effects and experiences if apparatus 100 comprises means for associating different stimulation units 110a, 110b, 110c with different (body) parts and/or regions of the user. For example, each hemisphere could thereby be associated with one hand of the user, assuming that he/she holds apparatus 100 with both hands.

It may also be advantageous to modify the onset times at which the stimulation units 110a, 110b, 110c are activated and thereby the velocity of the wave sensation. Assuming that the fingers of the user are placed substantially equidistantly and substantially perpendicular to the wave's direction of travel on apparatus 100, the vibro-tactile stimulation would be uniform in the sense that the user's fingers would be stimulated essentially one after the other, in equidistant intervals. However, the inventors have found that it may be advantageous if such uniform stimulation is precisely not used. Put differently, it may be advantageous if the velocity of the wave sensation varies, i.e., if the wave sensation speeds up or slows down. This also applies, e.g., when the fingers of the user are placed substantially along the wave's direction of travel on apparatus 100, such that the wave sensation travels along the user's fingers. To this end, the means for activating of apparatus 100 may be adapted to activate the stimulation units 110a, 110b, 110c, individually and/or in groups of two or more stimulation units, at N respective onset times T₁ . . . T_{N} throughout a cycle period T, wherein Nis an integer larger than one and wherein the onset times T₁ . . . T_{N} are not arranged substantially uniformly throughout the cycle period T. That is, the onset times T₁ . . . T_{N} are especially not arranged in uniform intervals substantially equal to T/N throughout the cycle period T. Rather, an onset time Tᵢ of the onset times T₁ . . . T_{N} may be substantially determined by a function that is nonlinear with respect to i. Relying on a function allows to determine onset times systematically as opposed to, e.g., randomly, thereby enhancing reproducibility. This increased reproducibility may in turn render stimulation more reliable and effective.

Onset times substantially given by any of the following functions have been found to result in particularly effective stimulation, yielding especially strong and long-lasting effects:

One or more onset times Tᵢ of the onset times T₁ . . . T_{N} may be substantially given by Tᵢ=T/N·(i−1)+T/2N·(1+(−1)ⁱ)·C, wherein −1<C<0, preferably −0.5≤C<0, most preferably −0.2≤C<0.

One or more onset times Tᵢ of the onset times T₁ . . . T_{N} may be substantially given by Tᵢ=T/N·(i−1)+T/2N·(1+(−1)ⁱ)·C, wherein 0<C<1, preferably 0<C≤0.5, most preferably 0<C≤0.2.

One or more onset times Tᵢ of the onset times T₁ . . . T_{N} may be substantially given by Tᵢ=T/N·(i−1)·C, wherein 0<C<1, preferably 0.75≤C<1, most preferably 0.5≤C<1.

One or more onset times Tᵢ of the onset times T₁ . . . T_{N} may be substantially given by Tᵢ=T/N·(i−1)·C, wherein 1<C<N/N−1.

One or more onset times Tᵢ of the onset times T₁ . . . T_{N} may be substantially given by Tᵢ=T/N·(i−1)+T·C, wherein C=0 for i=1 and −1/N<C<0 for i>1.

One or more onset times Tᵢ of the onset times T₁ . . . T_{N} may also be substantially given by Tᵢ=T/N·(i−1)+T·C, wherein 0<C<1/N.

One or more onset times Tᵢ of the onset times T₁ . . . T_{N} may be substantially given by Tᵢ=T/N·(i−1)+T·(N+1−i)·C, wherein C=0 for i=1 and −1/N·(N−1)<C<0 for i>1.

One or more onset times Tᵢ of the onset times T₁ . . . T_{N} may be substantially given by Tᵢ=T/N·(i−1)+T·(N+1−i)·C, wherein 0<C<1/N.

One or more onset times Tᵢ of the onset times T₁ . . . T_{N} may be substantially given by T₁=0 for i=1 and by Tᵢ=Tᵢ₋₁+T/N·C^{N+1-i} for i>1, wherein 0<C<1, preferably 0.75<C<1, most preferably 0.5<C<1.

One or more onset times Tᵢ of the onset times T₁ . . . T_{N} may be substantially given by T₁=0 for i=1 and by Tᵢ=Tᵢ₋₁+T/N·C^{N+1-i} for i>1, wherein 1<C and C^{N}−C/C−1<N.

When employing any of the above formulas, C may be determined using an optimization algorithm based on the (measured) effectiveness of the stimulation.

Any such pattern may furthermore take into account the precise position of the user's fingers—or any other (body) part and/or region of the user, for that matter—on apparatus 100. That is, apparatus 100 may comprise means (e.g., sensors) for establishing which stimulation units 110a, 110b, 110c are in contact with the user, and the means for activating may be adapted to activate (only) a subset of the stimulation units 110a, 110b, 110c that consists of stimulation units 110a, 110b, 110c that are established to be in contact with the user. That is, any such pattern may compensate for the fact that only a—not necessarily uniformly distributed—subset of the stimulation units 110a, 110b, 110c will usually be in contact with the user.

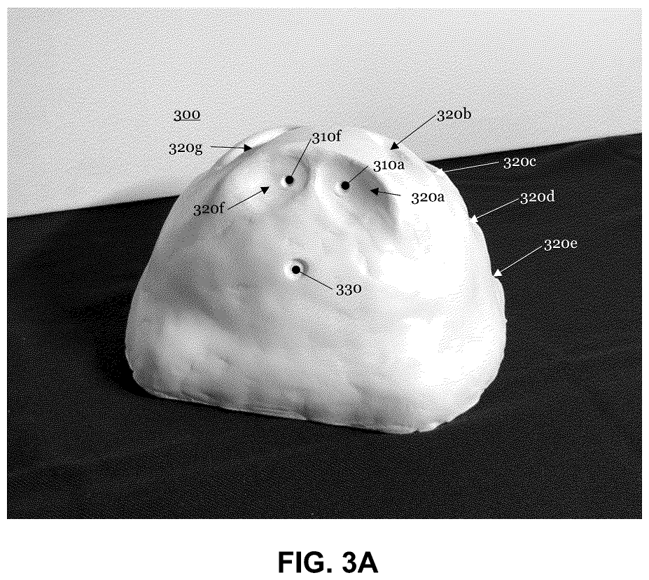

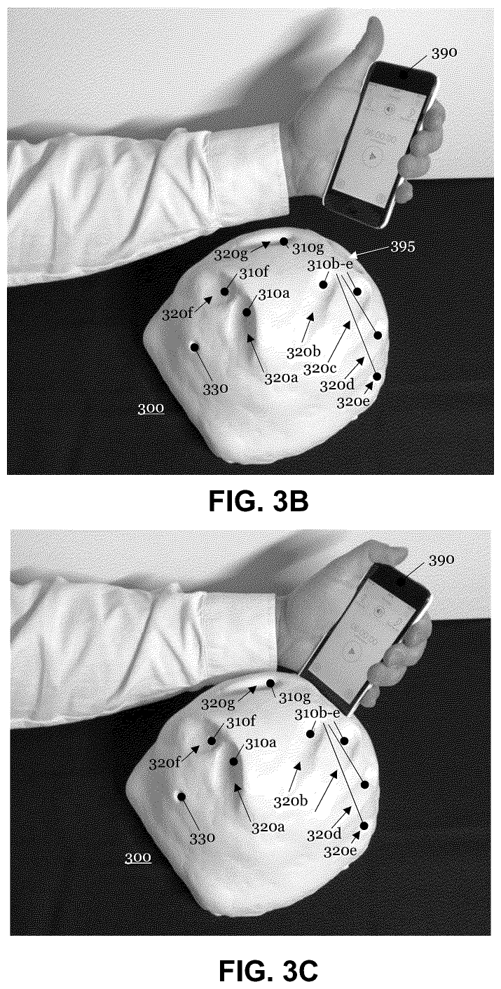

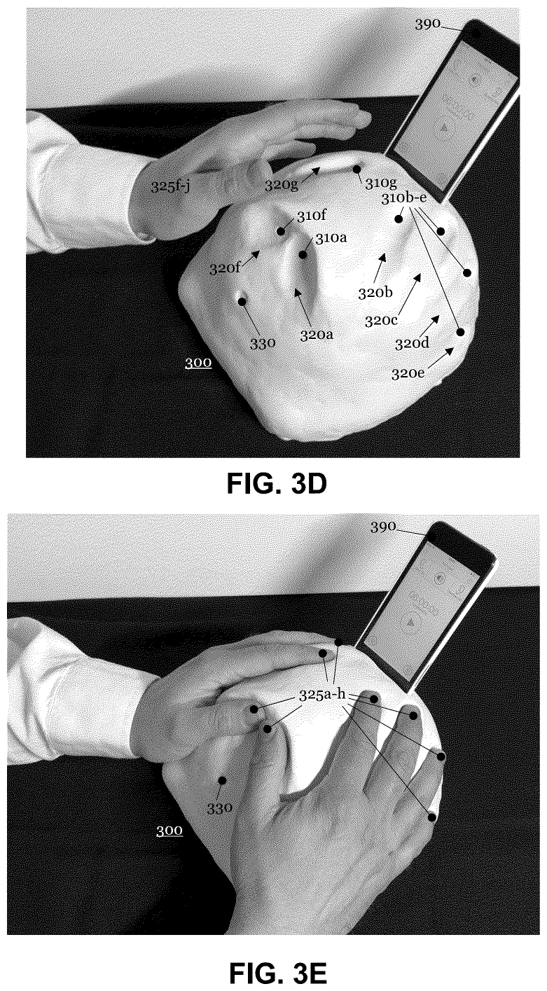

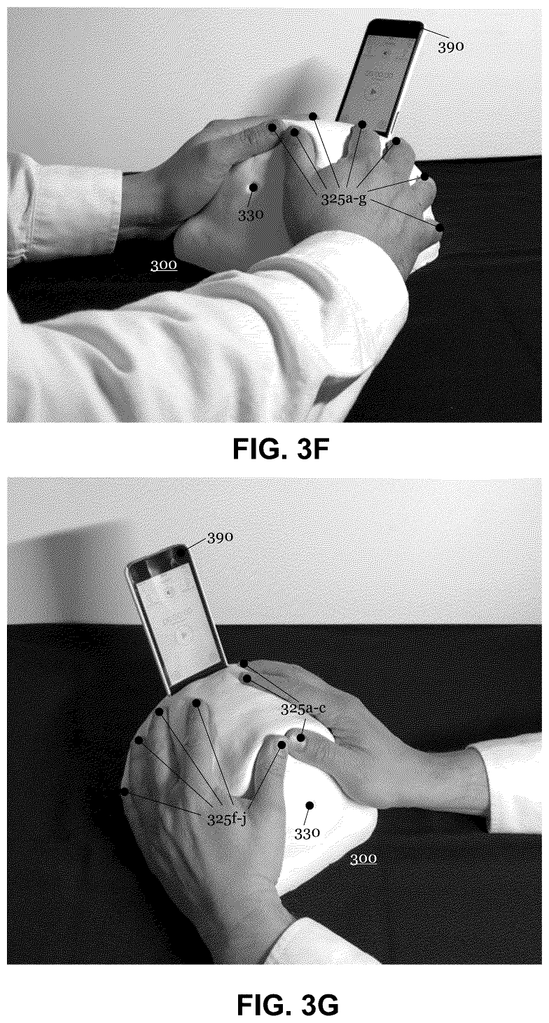

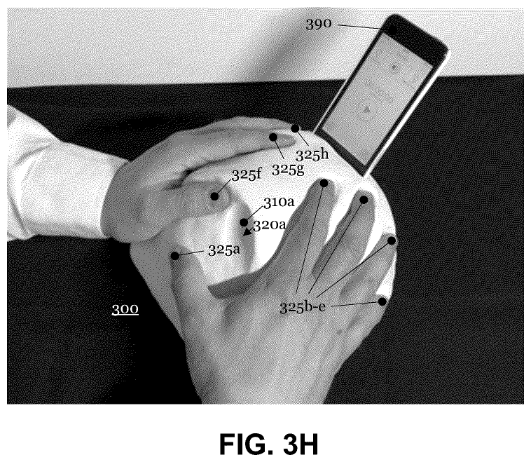

FIGS. 3A-3H show different views of another embodiment of the apparatus according to the present invention. The shown apparatus 300 has a generally ergonomic shape. In the example shown, the apparatus 300 has a generally half-spherical. The apparatus 300 is adapted to receive one or both hands of a user. In the example shown, apparatus 300 is adapted to receive both hands of a user. More specifically, apparatus 300 comprises a plurality of recesses 320a-320g adapted to receive the fingers and thumbs, in particular their tips 325a-325j, of the user (note that there may be separate recesses for tips 325i and 325j and/or the respective fingers and/or thumbs, as well; however, they are not visible throughout FIGS. 3A-3H and have hence not been provided with reference signs). More generally, the apparatus 300 may be modified to comprise a plurality of recesses adapted to receive some or all of the fingers and thumbs of one user hand, or both user hands.

Each of recesses 320a-320g comprises a respective stimulation unit 310a-310g, i.e., in each of the recesses 320a-320g, there is arranged a stimulation unit 310a-310g. More specifically, stimulation units 310a-310g are arranged within the recesses 320a-320g such that they are in contact with tips 325a-325j once they (and/or the respective fingers and/or thumbs) are placed within recesses 320a-320g.

Apparatus 300 may furthermore comprise means for initiating or allowing establishing a wireless connection, for example a button 330, e.g., a touch-sensitive button. In the embodiment of FIGS. 3A-3H, button 330 is arranged below recesses 320a and 320f adapted to receive (the tips 325a and 325f of) the thumbs of the user.

Moreover, in a further aspect, apparatus 300 may comprise an opening 395 which functions to receive a separate device 390 as described above, in this case a smartphone. In the embodiment of FIGS. 3A-3H, opening 395 is arranged at an opposite end of apparatus 300 with respect to recesses 320a and 320f and button 330. This may allow a display of separate device 390 to be visible to the user during use. Alternatively, instead of providing for an opening, apparatus 300 may also comprise other means for allowing to put the separate device 390 onto the apparatus 300, e.g. a cradle, or may also not be equipped with further means to hold the separate device 390. For example, in the latter case, separate device 390 may be place in a cradle next to apparatus 300.

The process of placing separate device 390 in opening 395 is illustrated in FIGS. 3B and 3C. FIG. 3D illustrate the process of placing the hands and fingers and/or thumbs (or their tips 325a-325j, respectively) of the user on apparatus 300.

FIGS. 3E-3G show different views of apparatus 300 in use, i.e., in a situation where both hands, i.e., all fingers and/or thumbs, in particular their tips 325a-325j, are placed on apparatus 300, i.e., in corresponding recesses 320a-320g. FIG. 3H illustrates the advantage of having button 330 arranged below recesses 320a and 320f adapted to receive (the tips 325a and 325f of) the thumbs of the user. As illustrated, this arrangement allows the user to simply remove one of his/her thumbs from the respective recess 320a or 320f and use it to operate button 330, notably without removing any other of his/her fingers or the respective other thumb from their respective recesses 320f or 320a, 320b-e and 320g-j.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. An apparatus for providing vibro-tactile stimulation for use in combination with a psychological intervention, the apparatus comprising:
a plurality of stimulation units, each adapted to provide vibro-tactile stimulation to a user upon activation;
a receiver configured to receive instructions as part of the psychological intervention; and
a controller configured to activate at least a subset of the plurality of stimulation units according to the instructions.

2. The apparatus according to claim 1, wherein the psychological intervention is provided by a computer program, and wherein the receiver is adapted to receive the instructions from the computer program.

3. The apparatus according to claim 2, wherein the computer program is executed by a separate device, and wherein the receiver is adapted to receive the instructions from the separate device.

4. The apparatus according to claim 2, wherein the apparatus comprises a processor configured to execute the computer program itself, and means for providing the psychological intervention to the user.

5. The apparatus according to claim 1, wherein the psychological intervention comprises elements of Mindfulness Based Cognitive Therapy (MBCT) and/or relaxation training.

6. The apparatus according to claim 1, further comprising a sensor configured to establish which stimulation units of the plurality of stimulation units are in contact with the user, wherein the controller is adapted to activate at least a subset of the plurality of stimulation units that consists of stimulation units that are established to be in contact with the user.

7. The apparatus according to claim 1, further comprising means for associating different stimulation units of the plurality of stimulation units with different parts of the user.

8. The apparatus according to claim 1, further comprising a surface layer covering at least a portion of the apparatus, wherein the surface layer:
is removable; and/or
comprises a rinseable and/or skin-friendly material.

9. The apparatus according to claim 1, further comprising an energy storage that is replaceable and/or rechargeable.

10. The apparatus according to claim 1, comprising between 2 and 500 stimulation units.

11. The apparatus according to claim 1, wherein the stimulation units are arranged at an average distance of 0.1 cm to 20 cm.

12. The apparatus according to claim 1, wherein the apparatus has a generally spherical shape with a diameter between 5 cm and 100 cm.

13. The apparatus according to claim 1, wherein the apparatus is in a shape of a pillow.

14. The apparatus according to claim 1, further comprising one or more recesses adapted to receive at least a tip of a finger and/or thumb of the user, wherein each of the one or more recesses comprises at least one stimulation unit of the plurality of stimulation units.

15. The apparatus according to claim 1, wherein the apparatus is at least partially hollow such that it is positionable over another object.

16. The apparatus according to claim 1, wherein the apparatus is in a shape of a glove.

17. The apparatus according to claim 1, wherein the controller is adapted to activate at least the subset of the plurality of stimulation units synchronously to generate a pulsing sensation for the user.

18. The apparatus according to claim 1, wherein the controller is adapted to activate at least one or more subsets of the plurality of stimulation units throughout a plurality of bursts;
wherein, within a burst, the one or more subsets of the plurality of stimulation units are activated at a frequency in the range of 10 Hz to 300 Hz; and
wherein there are 100 ms to 2000 ms between the bursts.

19. The apparatus according to claim 1, wherein the controller is adapted to successively activate at least the subset of the plurality of stimulation units, individually and/or in groups of two or more, so as to generate a sensation of a wave travelling across the apparatus for the user.

20. The apparatus according to claim 1, wherein the controller is adapted to activate the subset of the plurality of stimulation units, individually and/or in groups of two or more, at N respective onset times T₁ . . . T_{N} throughout a cycle period T, wherein N is an integer larger than one and wherein the onset times T₁ . . . T_{N} are not arranged substantially uniformly throughout the cycle period T.

21. A tangible, non-transitory computer-readable medium comprising a computer program for providing a psychological intervention for use in combination with an apparatus for providing vibro-tactile stimulation, the computer program comprising instructions which, when the computer program is executed by a computer, cause the computer to output instructions to the apparatus as part of the psychological intervention.

22. A system for providing vibro-tactile stimulation in combination with a psychological intervention, the system comprising:
the apparatus according to claim 1; and
a computer program comprising instructions which, when the computer program is executed by a computer, cause the computer to output instructions to the apparatus as part of the psychological intervention, or a separate device configured to execute the computer program.

23. A method for providing vibro-tactile stimulation for use in combination with a psychological intervention, the method comprising:
providing instructions to a user as part of the psychological intervention; and
activating at least a subset of a plurality of stimulation units, each adapted to provide vibro-tactile stimulation to the user upon activation, according the instructions.
